(19) United States
(12) Patent Application Publication
(10) Pub. No.: US 2004/0137039 A1
(43) Pub. Date: Jul. 15, 2004

(54) Methods for controlled release of molecules from layered polymer films

(75) Inventors: Svetlana A. Sukhishvili, Maplewood, NJ (US); Eugenia Kharlampieva, Jersey City, NJ (US); Vladimir A. Izumrudov, Moscow (RU)

Correspondence Address: Ralph W. Selitto, Jr., McCarter & English, Four Gateway Center, 100 Mulberry Street, Newark, NJ, 07102, US

(73) Assignee: Trustees of Stevens Institute of Technology

(21) Appl. No.: 10/624,993

(22) Filed: Jul. 22, 2003

Related U.S. Application Data
(60) Provisional application No. 60/397,960, filed on Jul. 22, 2002.

(51) Int. Cl. A61K 9/70

(52) U.S. Cl. 424/443

(57) ABSTRACT

Low molecular weight molecules are selectively released from a layered polymer film having a net excess charge by introducing at least one other type of molecule that binds reversibly to the film and thereby reduces the net excess charge. Oligomeric and polymeric molecules, whether synthetic or natural, are selectively and reversibly released from the layered polymer film in response to variation in ionic strength in the environment of the film. Such molecules are also selectively and reversibly released from the layered polymer film in response to changes in the pH in the environment of the film.

### CROSS-REFERENCE TO RELATED APPLICATION

This application claims the benefit of U.S. Provisional Patent Application No. 60/397,960, filed Jul. 22, 2002.

The subject matter of this application was funded in part by the National Science Foundation (Grant no. DMR-0209439). The government of the United States of America may have certain rights in this invention.

### FIELD OF THE INVENTION

The present invention relates to the controlled release of molecules from layered polymer films.

### BACKGROUND OF THE INVENTION

Polymer films find wide-ranging applications from coatings to drug delivery materials. A recently introduced technique of making polymer films involves the sequential deposition and self-assembly of polymer layers from solution. The best known examples of layer-by-layer self-assembly rely on electrostatic attraction of polymers of opposite charges, but hydrogen bonding and Van der Waals interaction may be also used to produce such films. The formation of ultrathin self-assembled films by means of electrostatic attraction is described, for example, in U.S. Pat. No. 5,208,111. Techniques to immobilize proteins at the surface of a multilayer film by means of consecutive alternating adsorption of molecular layers of proteins and polyelectrolytes bearing opposite electric charges are disclosed in WIPO Publication WO 96/30409 and can be also found in many journal publications. The layers of film may be built up from solutions, and molecules such as drugs, dyes and other molecules, may be absorbed into the multi-layer film, after the film has been formed. Alternatively, oligomeric or polymeric molecules, such as natural and synthetic polypeptides, oligo- and polynucleotides and other, similar types of molecules may be assembled within the multilayer films by means of sequential adsorption. In many applications, it is desirable to provide for the controlled release of molecules that are absorbed within the film. In cases where the layers of the film are built up from polymers with ionizable functional groups, the charge of which depends on pH, such release requires that the net charge of some or all of the layers be altered to overcome the electrostatic attraction that holds the molecules within the film. One known method for releasing the absorbed molecules from the film is by the application of an external electric field. This method has been described in literature for the case of the films which are not produced by sequential self-assembly (see, for example, X. Sun, B. Lin, et al, “pH and potential-sensitive film of polyaniline for drug release”, Kexueban 2000, 21, 24-27; and M. Hepel, J. Hepel “Controlled binding and electrorelease of inorganic cations and drugs from composite polymer films”, Polym. Mater. Sci. Eng., 1994, 71, 717-718). Another method for reducing the net charge of the self-assembled polymer layers, thereby releasing absorbed molecules therefrom, is to change the pH of the external solution. Such use of pH-response to release biologically active molecules has been described for polymer coatings that do not contain layered nanostructure (see, e.g., U.S. Pat. No. 6,306,422; U.S. Patent Application No. 2003/0031699 by Antwerp et al). U.S. Pat. No. 6,068,853 also describes the use of pH-oscillating chemical reaction to achieve pulsate delivery of bioactive agents is described). Other references describe the release of low molecular weight molecules from polymer films in response to changes in the pH of the film's ambient environment (see, e.g., A. J. Chung and M. F. Rubner, “Methods of Loading and Releasing Low Molecular Weight Cationic Molecules in Weak Polyelectrolyte Multilayer Films”, Langmuir 2002, 18, 1176; S. A. Sukhishvili and S. Granick, Layered, Erasable, Ultrathin Polymer Films, J. Am. Chem. Soc. 122, 9550 (2000); and S. A. Sukhishvili and S. Granick, Layered, Erasable Polymer Multilayers Formed by Hydrogen-Bonded Sequential Self-Assembly, Macromolecules 35, 301 (2002)).

The present invention provides two general approaches for the triggered release of molecules from multilayer polymer films that differ from those described in the prior art. For small molecules, the triggering mechanism of release is the adsorption of macromolecules on the outermost layer of the multilayered film. The application of an electric field or external pH change are not part of the triggering event for the release of such small molecules. In the case of oligomeric and polymeric molecules, which are self-assembled within the multilayer film, the disclosed method provides the selective, reversible and controllable release of one of the components from the films as the external pH or ionic strength of the external solution is varied while providing little to no release of the other molecular components.

### SUMMARY OF THE INVENTION

It is an object of the present invention to provide a method of releasing low molecular weight molecules, such as drugs, dyes, or other molecules, from a layered polymer film having a net excess charge, by introducing to the system at least one other type of molecule that binds reversibly to the film and thereby reduces the net excess charge.

Another object of this invention is to provide a method of selectively and reversibly releasing oligomeric and polymeric molecules, such as natural and synthetic polypeptides, oligo- and polynucleotides or other molecules having plurality of charges, from a layered polymer film, in response to variation in ionic strength of the environment of the film.

Yet another object of this invention is to provide a method of selectively and reversibly releasing oligomeric and polymeric molecules, such as natural and synthetic polypeptides, oligo- and polynucleotides or other molecules having plurality of charges, from a layered polymer film system, in response to changes in the pH of the environment of the film.

Brief Description the Drawings

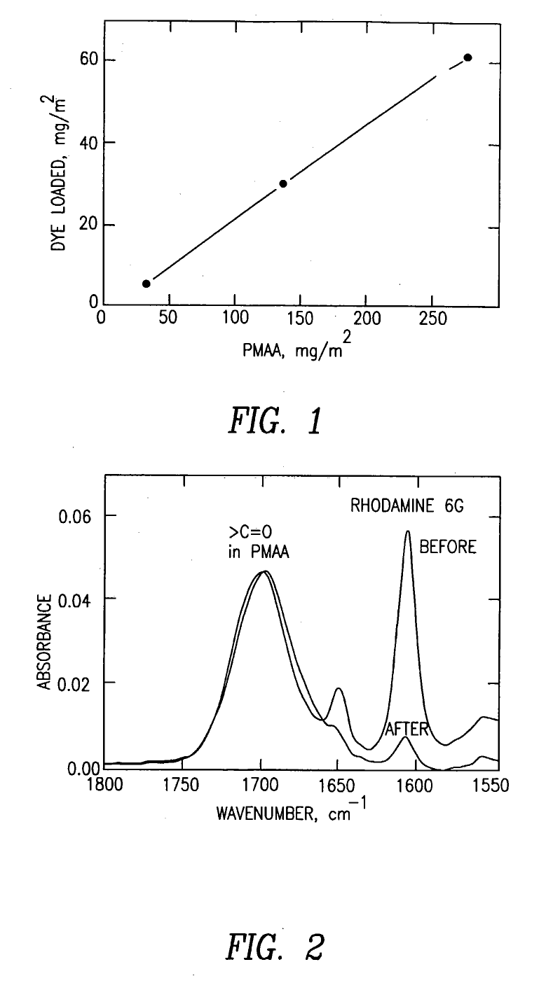

FIG. 1 is a plot of the amount of dye loaded into a multilayer film of the present inventiont plotted against the amount of poly(methacrylic acid) [PMMAA] in the film at equilibrium;

FIG. 2 is a plot of the infrared absorbance of a multilayer film of the present invention against wavenumber, before and after the release of Rhodamine 6G has been triggered by PMMAA adsorption;

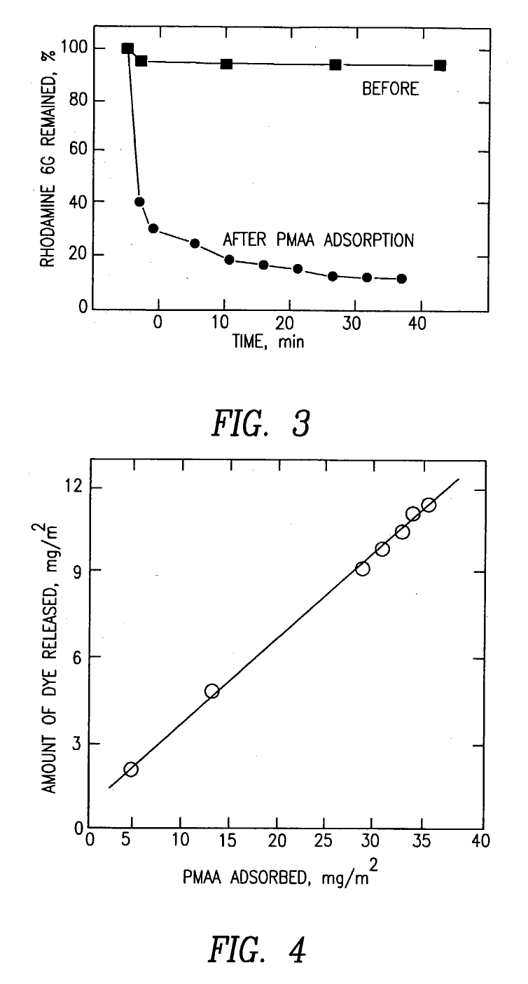

FIG. 3 is a plot against time of the amount of Rhodamine 6G remaining in the multilayer film of FIG. 2, before and after PMAA adsorption;

FIG. 4 is a plot of the amount of dye released from the multilayer film of FIG. 2 plotted against the amount of PMAA absorbed on the surface of the film;

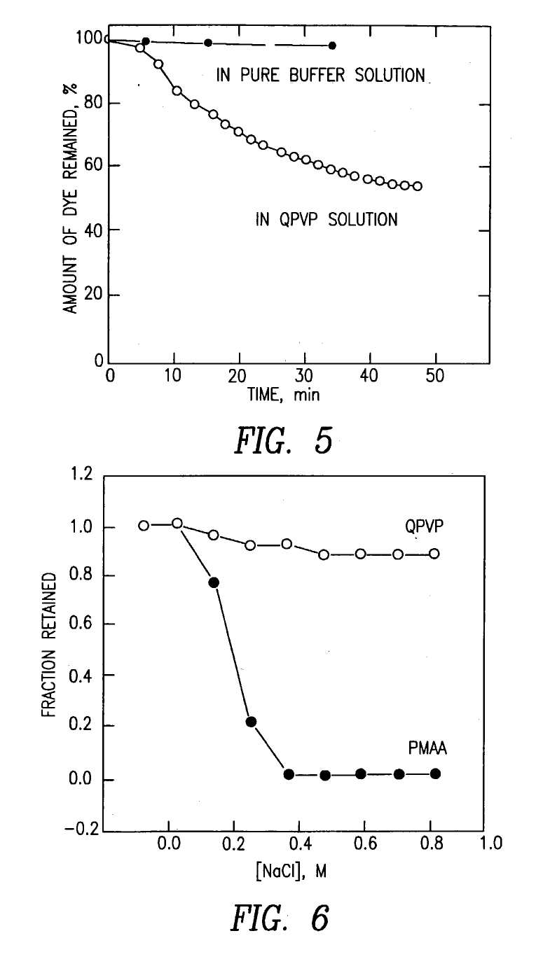

FIG. 5 is a plot against time of the amount of Bromophenol Blue remaining in a multilayer film of the present invention, after treatment with pure buffer solution and after QPVP adsorption;

FIG. 6 is a plot of the fractions of PMAA or quaternized poly-4-vinylpyridine [QPVP] retained within a QPVP/PMAA film as a function of the ionic strength in the aqueous environment of the film;

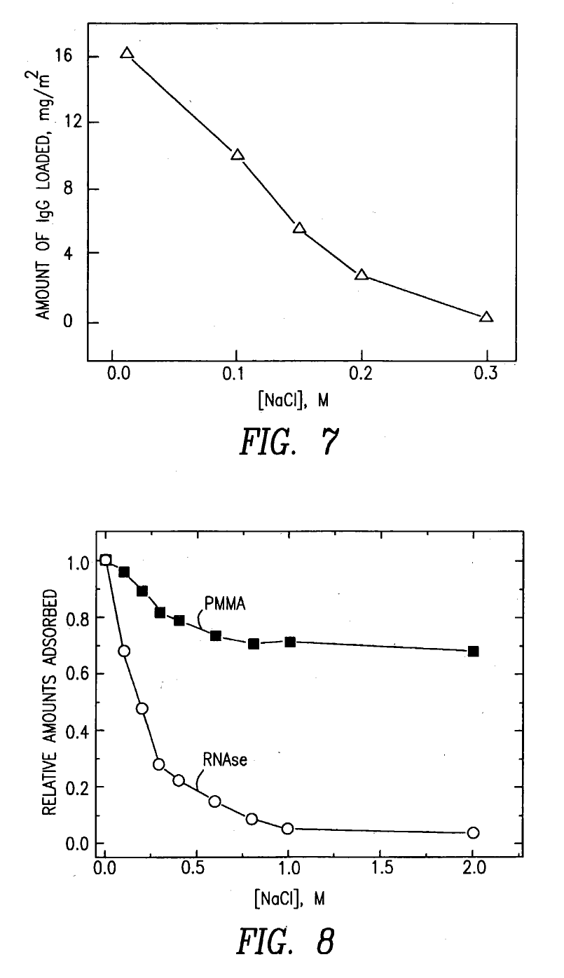

FIG. 7 is a plot of the amount of IgG absorbed within a QPVP film as a function of the ionic strength in the aqueous environment of the film; and

FIG. 8 is a plot of a fraction of ribonuclease [RNAse] or PMAA remaining within a RNAse/PMAA film as a function of the ionic strength in the aqueous environment of the film.

### DETAILED DESCRIPTION OF THE INVENTION

In a preferred embodiment, the present invention provides a method of releasing low molecular weight molecules, such as drugs, dyes, or other molecules, from a layered polymer film system, by including at least one type of molecule having plurality of charges and/or hydrogen-donating or hydrogen-accepting moieties in a solution that is in contact with the polymer film carrying the absorbed molecules.

As demonstrated in Examples 1 and 2, below, the low-weight molecules are absorbed in a self-assembled layered polymer film. The self-assembly process may involve the formation of hydrogen bonds and/or electrostatic attraction between polymers in adjacent layers. The charged molecules, which may be drugs or dyes or the like (hereinafter Molecule A), can be absorbed or ‘trapped’ within one or more layers of the film during formation, or absorbed or otherwise added to the film after the film is produced. The electrostatic attraction between Molecule A to the excess charge existing in one or more layers of the polymer film causes Molecule A to be trapped within the film. Reducing the excess charge level in the films can reduce the affinity of Molecule A to the film. When a solution containing a different molecule (Molecule B), which carries a charge of the same sign as the excess charge in the polymer film, contacts the film and Molecule B absorbs at the film surface, the amount of excess charge in the film decreases due to local electrostatic effects, which in turn causes the controlled release of Molecule A from the film.

Furthermore, the amount of charge provided to the film surface upon adsorption of Molecule B controls the quantity of Molecule A that is released from the film. Unlike previous release mechanisms, alteration of the environmental conditions, such as changes in solution pH or application of electric field, are not required for the release of Molecule A. This release is sensitive merely to the presence of Molecule B on the outermost absorbed layer of the film.

The polymers used to form the layered polymer films in the present invention include at least one polymer having a charge-forming group that can be modulated between the charged and uncharged states, thereby altering the net charge of the polymer layer. That polymer, and the others in the film, may also include any or all of the following three moieties: a) a group with a permanent charge that is complementary to the charged state of the charge-forming group; b) a hydrogen bond donor; or c) a hydrogen bond acceptor. Charge-forming groups are moieties that can develop a charge when exposed to different environmental conditions, such as pH, a change in ionic strength or exposure to an electric field. Examples of charge-forming groups include acid or base moieties.

Combinations of polymers which have utility in the present invention may be broadly classified into three Groups:

1) Polymers of Group 1 include polymer 1*-polymer 1** pairs, where polymer 1* is a polymer containing charge-forming groups, preferably, a weak polyacid, and hydrogen bond donors and/or hydrogen bond acceptors. Polymer 1** is a polymer containing hydrogen bond donors and/or hydrogen bond acceptors that are complementary to hydrogen-bonding moieties of polymer 1*. In this Group, a layer of polymer 1* adheres to a layer of polymer 1** through hydrogen-bonding. Polymer 1* is not completely ionized under the conditions at which the film is formed and, therefore, the net charge of the layered film can be modulated by changing the environmental pH. For example, in cases where polymer 1* is a weak polyacid, increasing the environmental pH results transforms the acidic moieties to their basic form, creating an excess amount of negative charges in the layered film.

2) Polymers of Group 2 include polymer 2*-polymer 2** pairs, where polymer 2* is a strong polyacid containing charge-forming groups or a polyacid with permanent charges. Polymer 2** is a weak polybase with chargeable groups. In this Group, a layer of polymer 2* adheres to a layer of polymer 2** through electrostatic bonding. Films comprising Group 2 polymer pairs are preferably formed at a higher pH than the films comprising polymer pairs of Groups 1 or 3. The polybase is not completely ionized in the conditions at which the film is formed. Therefore, the films typically have a net positive charge and are pH-sensitive, with the net charge becoming more positive as the environmental pH decreases.

3) Polymers of Group 3 include polymer 3*-polymer 3** pairs, where polymer 3* is a weak polyacid containing charge-forming groups. Polymer 3** is a polybase with permanently charged and/or chargeable groups. Similar to the Group 2 polymers, a layer of polymer 3* adheres to a layer of polymer 3** through electrostatic bonding. Films comprising Group 3 polymer pairs typically have a net negative charge and are pH-sensitive, with the net charge becoming more negative as the environmental pH increases, as described for the polymers of Group 1.

With reference to polymers of Group 1, polymer 1* may be a polymer of the group comprising, but not limited to, polycarboxylic acid such as polyacrylic acid, polymethacrylic acid, polyitaconic and polycrotonic acid, polynucleotides such as poly(adenylic acid), poly(uridylic acid), poly(cytidylic acid) and poly(inosinic acid), polymers of vinyl nucleic acids such as poly(vinyladenine), and polyamino acids such as polyglutamic acid. Polymer 1** is may be a member of the group comprising, but no limited to, polyalcohols such as poly(vinyl alcohol), polyethers such as poly(ethylene oxide), poly(1,2-dimethoxyethylene) and poly(vinylmethyl ether), polyketones and polyaldehydes such as poly(vinyl butyral) and poly(N-vinyl-2-pyrrolidone), polyacrylamides such as polyacrylamide, polymethacrylamide and poly(N-isopropylacrylamide), and copolymers thereof.

With reference to the polymers of Group 2, polymer 2* may be a polyacid of the group comprising, but not limited to, polycarboxylic acid such as polyacrylic acid, polymethacrylic acid, polyitaconic and polycrotonic acid, polynucleotides such as poly(adenylic acid), poly(cytidylic acid), poly(uridylic acid) and poly(inosinic acid), polymers of vinyl nucleic acids such as poly(vinyladenine), and polyamino acids such as polyglutamic acid; or polyacids containing permanently charged groups such as poly(styrene sulfonic acid), poly(vinyl sulfonic acid) and poly(vinyl phosphoric acid). Polymer 2** is a polybase of the group comprising, but not limited to, partially quaternized poly(vinyl pyridines), poly(imidazoles) and polyamines such as poly(4-amino)styrene, polyethylene imines, poly(allyl amine) and poly(vinyl amine).

With reference to the polymers of Group 3, polymer 3* may be a polyacid of the group comprising, but not limited to, polycarboxylic acid such as polyacrylic acid, polymethacrylic acid, polyitaconic and polycrotonic acid, polynucleotides such as poly(adenylic acid), poly(uridylic acid), poly(cytidylic acid), poly(uridylic acid) and poly(inosinic acid), polymers of vinyl nucleic acids such as poly(vinyladenine), and polyamino acids such as polyglutamic acid. Polymer 3** is a polybase of the group comprising, but not limited, to quaternized poly(vinyl pyridines), quaternized poly(imidazoles), poly(dimethyldiallyl) salts, quaternized poly(diaminoethoxy methacrylates) and poly(diaminoethoxy acrylates) and polyamines such as poly(4-amino)styrene, polyethylene imines, poly(allyl amine) and poly(vinyl amine).

Abbreviations of the various polymer names and other chemical names used hereinbelow are listed in Table 1, below.

TABLE 1
Abbreviations of Chemical Names Used Herein

| Abbreviation | Chemical name |
|---|---|
| PMAA | polymethacrylic acid |
| PAA | polyacrylic acid |
| PEO | polyethylene oxide |
| PVPON | Polyvinylpyrrolidone |
| PVA | poly(vinyl amine) |
| PALA | poly(allyl amine) |
| QPVP | quaternized poly-4-vinylpyridine |
| PTMMAEA | poly(N,N,N,-trimethyl- |
| | 2-methacryloylethylammonium)bromide |
| PDADMA | poly(diallyldimethyl ammonium)chloride |
| PSS | poly(styrene sulfonic acid) |
| PVPh | poly(vinyl phosphoric acid) |
| PVS | poly(vinyl sulfonic acid) |
| IgG | Immunoglobuline |
| RNAse | Ribonuclease |
| Lys | Lysozyme |

Molecule A can be of any chemical structure, as long as it carries a charge that is of the opposite sign to the sign of the excess charges in the polymer film and as long as it can be dissolved in a solvent that is will not dissolve or degrade the polymer film. Aqueous solvents are preferred, but layered films within the scope of the present invention can also be created and operated in non-aqueous mixtures, as will be understood by those skilled in the relevant arts. Examples of suitable Molecule A include dyes and bioactive agents. The bioactive agents can be any physiologically or pharmacologically active substance that is soluble in water. Such agents include drugs, proteins, peptides, genetic materials, nutrients, vitamins, food supplements, fertility inhibitors, fertility promoters, vitamins, nutrients, or the like.

On the basis of the foregoing discussion, it should be understood that Molecule A suitable for use with polymer pairs of Groups 1 and 3 include molecules that carry positive charges or groups that form positive charges. Molecule A suitable for use with polymer pairs of Group 2 (e.g., the polymer pairs of Examples 2 and 3, hereinbelow) are those that carry negative charges or groups that form negative charges.

Molecule A that contain positive charges, or groups that form positive charges, include antibiotics such as pivampicillin and cephaloridine; antiinflammatory agents such as glaphenine; anesthetics such as benzocaine, procaine and piridocaine; hormones, neutrotransmitters and humoral factor such amphetamine and meparfynol; antidepressants and tranquilizers such as etryptamine, methpimazine and pipamazine; antispasmodic agents such as methantheline bromide, propanetheline bromide and fenethylline; miscellaneous drugs such as hycanthone; antihypertensive agents such as dihydralazine and bretylium tosylate; anesthetics and central nervous system stimulants such as neostigmine, ephedrine, oxyfedrine, levonordefrine, amphetamine, tranylcypromine, fencamfine and hydroxyamphetamine; antidepressants such as phenelzine and pheniprazine; antidiabetic agents such as phenformin; antibiotics such as ethionamide, protonsil, sulfanilamide and sulfanilamide derivatives; antiinfective agents such as chlorazanil, aminophenazole, trimethoprim, pyrimethamine, primaquine and sontoquine; analgetics such as phenazopyridine; hypotensive agents such as minoxidil; obesity control agents such as phentermine and chlorphentermine; diuretic agents such as chlorazanil, aminotetradine, amiloride and amisotetradine; anticoccidial drugs such as amprolium; anthelmentic agents such as dithiazinine. Further examples include neurotoxins and vitamins such as thiamine (B₁), nicotinamide (B₃), pyridoxamine (B₆).

Molecule A that contain negative charges, or groups that form negative charges, include antiinflammatory agents such as aspirin, fenamic acids (flufenamic and mefanamic acids), ibuprofen, flubiprofen, naproxen and indomethacin; anesthetics such as ecgoninic acid; antidepressants such as dibenzoxepins; hormones, neutrotransmitters and humoral factor such as prostoglandines (dinoprost, PGE₁, PGF_{1α}, PGF_{2α} and PGE_{2,}), estrogens (methallenestril); enzyme inhibitors such as nodularin and its synthetic derivatives cyclo[-(3S,E)-3-phenylethenyl-3-aminopropanoyl-α-(R)-Glu-α-OH-γ-Sar-(R)-Asp-α-OH-β-(S)-Phe-] and cyclo[-(2S,3S,E)-2-methyl-3-phenylethenyl-3-aminopropanoyl-β-(R)-Glu-α-OH-γ-Sar-(R)-Asp-α-OH-Pβ-(S)-Phe-]; antibiotics such as acephylline, carbencillin, cephalothin, nafcillin, methicillin and penicillin G; antihypertensive agents such as bretylium tosylate; muscle relaxants such as phenyramidol; diuretic agents such as ethacrynic acid and probenecid. Further examples include vitamins such as pantothenic acid (B₅) and cofactors such as biotin and trombomodulin.

Molecule B can be of any structure, as long as it absorbs to the polymer film surface through hydrogen and/or electrostatic interactions or can be self-assembled with the polymers 2*, 2** or 3**, and can be dissolved to useful concentrations in the solvent. Examples of Molecule B include any synthetic or natural molecule, including bioactive agents. Such agents include synthetic water-soluble polymers, nucleic acids, proteins and synthetic polypeptides. It should further be understood that Molecule B suitable for use with polymer pairs of Group 2 include molecules that carry positive charges. Molecule B suitable for use with polymer pairs of Group 1 include molecules that carry negative charges and form hydrogen bonds with the polymer film surface. Molecule B suitable for use with polymer pairs of Group 3 include molecules that carry negative charges. Molecule B which contain negative charges include, for example, synthetic polycarboxylic acids, alginic acid and proteins. Examples of Molecule B that contain positive charges include, for example, synthetic polycations; basic growth factors such as fibrinoblast growth factor-2 (FGF2) and insulin-like growth factor IGF-I, spermine and chitosane. Examples of Molecule B suitable for inclusion with polymer pairs of Group 3 include molecules that contain negative charges, such as synthetic polycarboxylic acids such as poly(styrenesulfonic acid) and poly(phosporic acid); proteins such as albumins and main soy protein; heparin-binding proteins; acidic growth factors such as fibrinoblast growth factor-1 (FGF1) and insulin-like growth factor IGF-II; tissue-type plasminogen activators (t-PA) used in thrombolitic therapy such as monteplase; cofactors such as heparin cofactor II hyaluronic acid, heparin and DNA and RNA molecules.

Examples of substrate materials, comprising monolithic solids or particles, which may be coated with the layered polymer films of the present invention, include polymers such as polyethylene and fluorocarbons (e.g., TEFLON), ceramics such as glass or alumina, semiconductors such as silicon or germanium, and minerals such as mica.

According to the present invention, a layered polymer film is coated onto a surface of such a substrate; an agent, such as a member of Molecule A, is absorbed by the layered polymer film; and the agent is released at a later time in response to the specific or non-specific adsorption of the charged molecules, such as a member of Molecule B, to the polymer film surface. Since the foregoing methodology is operative above and below pH 7, certain criteria are to be considered to determine whether to operate above or below pH 7. The determining factor is that the release of the absorbed molecules from the film should be carried out at pH values at which a fraction of the ionizable groups in the weak polyacid (case 1), or the weak polybase (case 2), is NOT ionized. The pH at which this occurs depends on the pK of the polyacid and or polybase that is included in the layered film and, additionally, on the strength and nature of the interactions of the polyacid or polybase with other polymer components of the system. For example, the pK of PMAA in solution is about 6. However, the ionization of PMAA within the film will be suppressed if it interacts with a hydrogen-bond acceptor, and will be enhanced if it interacts with a polybase by forming ionic pairs, thereby altering the pK of the PMAA. Stated in more general terms, the pK of a polyacid or a polybase in the layered polymer film will differ from its value in solution. As a first approximation, the pH range is simply determined from the intrinsic ionization properties (pK) of the weak acidic and weak basic groups as they exist in the film. The preferred operative pH range of the layered film is limited to a narrow range of pH values around the pK value of the moiety in the film, under which pH values the largest ionization changes will be observed.

In another embodiment of this invention, Molecule B is sequentially self-assembled with polymers 2*, 2** or 3** by means of electrostatic adsorption and is preferentially released from the layered polymer film when the ionic strength of solution is increased. In this embodiment, Molecule B preferably is any synthetic or natural molecule including bioactive agents. Examples 3-5 and FIGS. 6 and 8 present results showing that, in this embodiment, virtually all of Molecule B is released from the layered film, while little to none of the polymer 2*, 2** or 3** is released. Without being bound by a particular theory, it appears that such asymmetric releases of Molecule B occur because polymers 2*, 2** or 3** are stabilized at the surface by hydrogen bonding (as in the case of PMAA) or salt out of the film when ionic strength is increased (as in the case of QPVP). Such asymmetric releases leave behind a polymer layer containing large amounts (from 10 to 50 mg/m2) of a surface-bound polyelectrolyte of one type (i.e., polymer 2*, 2** or 3*).

In still another embodiment of this invention, molecule B (preferably, any synthetic or natural molecule including bioactive agents) is sequentially self-assembled with polymers 2*, 2** or 3** by means of electrostatic adsorption or hydrogen bonding and is selectively released from the layered polymer film in response to changes in pH in the environment of the film. Results of such triggered releases are presented in Examples 6 and 7, where it can be seen that Molecule B is reversibly released in preference to the polymer from group 2*, 2** or 3**. Without being bound by a particular theory, it appears that such asymmetric releases of Molecule B occur because the environmental pH change creates an excess charge of the same sign as the charge of Molecule B. This excess charge could be created within Molecule B and/or polymers 2*, 2** or 3**, resulting in a controlled release of Molecule B from layered self-assembled polymer-film, with the amount released being proportional to the change in the amount of excess charge.

The following illustrative examples are intended to demonstrate the application of the embodiments of the invention that are discussed hereinabove to certain representative polymers and members of Molecule A and Molecule B. The Examples are not intended to limit the scope of the invention in any way.

### EXAMPLE 1

### Polymers of Group 1

The general procedures for forming and characterizing the layered polymer film described in this Example were also used in Examples 2-7. The adsorption and ionization of pyridine rings and carboxylic groups in the polymers was quantified by in-situ Fourier transform infrared spectroscopy in attenuated total reflection (FTIR-ATR). The experiments were performed in D₂O buffered solutions using the flow-through liquid cell.

Prior to deposition, the surface of a silicon (Si) crystal was modified by a primer layer to enhance the adhesion of polymers to the Si crystal substrate. In particular, the surface was first modified by allowing QPVP to absorb from an 0.1 mg/ml solution in D₂O at pH 9.2 (0.01 M borate buffer). After waiting 30 minutes, the amount of QPVP absorbed reached a saturated value of about 1.5 mg/m², and the polymer solution was replaced by a pure buffer. The foregoing procedure covered the surface of the Si crystal with a layer of cationic molecules carrying permanent electrical charge. A solution of PMAA (0.1 mg/ml in the same buffer solution) was added. The saturated amount of polycarboxylic acid deposited at this step, about 0.5-0.7 mg/m², was consistent with a charge compensation mechanism of the adsorption. This substrate (containing the 2-layer pretreatment) was used for multilayer polymer deposition and a buffer solution containing 0.01 M HCl was injected into the liquid cell.

Multiple layers of PEO (MW=200,000) were then deposited in alternating sequence with layers of PMAA (MW=150,000) on the surface of the modified Si crystal, so that the PMAA layers were uncharged. The procedure used was to allow a 0.1 mg/ml solution of PEO to absorb to the surface of the modified Si crystal, at pH 2; for 40 min, then replace the polymer solution by a buffer without polymer. PMAA was then deposited on top of the PEO layer in a similar manner. The deposition cycle was repeated until the desired number of polymer layers had been deposited. An 11-layer PMAA/PEO film, having a thickness of 134 nm, with PEO in the outermost layer, was formed.

The solution pH was then changed by contacting the surface of the PMAA/PEO film with 0.01 M phosphate buffer solution at pH 4.2. At this pH, the PMAA became 6% ionized. Rhodamine 6G dye was then absorbed into the PMAA/PEO film by contacting the surface of the PMAA/PEO film with a solution of 0.5 mg/ml Rhodamine 6G dye in the same buffer. The film was allowed to absorb the dye from solution for 1 hour. The Rhodamine 6G solution was then replaced by a buffer at pH 4.2 without Rhodamine 6G. The representative spectra of the PMAA/PEO film before and after addition of Rhodamine 6G are shown in FIG. 2. The dye content within the film was then monitored as a function of time. There was no significant desorption of the dye from the film for 1 hour (FIG. 3). The amount of dye absorbed was in 1:1 stoichiometric ratio with the amount of ionized groups in the film.

The buffer solution was then replaced by a 0.1 mg/ml PMAA solution at pH 4.2. Fast release of the dye was observed (i.e., 80% of the dye was released within first 2 minutes) (see FIG. 3). The release rate was found to be limited by the rate of PMAA adsorption. In addition, the amount of the dye released is proportional to the amount of PMAA absorbed (see FIG. 4).

In accordance with the above procedures, Rhodamine 6G was the absorbed into the same film at a different pH (i.e, pH 3.8) and the pH adjusted to release the dye. The degree of uptake and release of Rhodamine 6G by the film at pH 4.2 and pH 3.8 are shown in Table 2.

TABLE 2
Absorption and Release of Rhodamine 6G
in a Layered PMAA/POE Film

| | | Amount of | Percent of |
|---|---|---|---|
| | Percent of | Rhodamine 6G | Rhodamine 6G |
| pH | COOH ionized | absorbed, mg/m² | released |
| 3.8 | 3 | 23 | 17 |
| 4.2 | 6 | 31 | 40 |

In accordance with the above procedures, the experiment described above, was performed with PMAA/PEO films of various thicknesses, assembled according to the same procedure described above, at pH 4.2 and pH 3.8. As demonstrated by the data in Table 3, at a given pH, the amount of the dye loaded (absorbed) was linearly proportional to the film thickness (FIG. 1), suggesting the absorbed dye was uniformly distributed within the film.

TABLE 3
Absorption and Release of Rhodamine 6G in
Layered PMAA/POE Films of Various Thicknesses

| | | | | Amount of | Percent of |
|---|---|---|---|---|---|
| | | Total film | Percent of | Rhodamine | Rhodamine |
| | Number of | thickness, | COOH | 6G absorbed, | 6G |
| PH | layers | nm | ionized | mg/m² | released |
| 4.2 | 3 | 24 | 6 | 8 | 87.6 |
| 4.2 | 7 | 70 | 6 | 20 | 68 |
| 4.2 | 9 | 134 | 6 | 31 | 40 |
| 4.2 | 13 | 238 | 6 | 48 | 33 |
| 3.8 | 5 | 36 | 3 | 5 | 47.8 |
| 3.8 | 11 | 175 | 3 | 30 | 22 |
| 3.8 | 15 | 337 | 3 | 57 | 14.4 |

Similar results were obtained in layered polymer films having PVPON in place of PEO, and PM in place of PMAA.

### EXAMPLE 2

### Polymers of Group 2

QPVP was prepared by reacting poly-4-vinylpyridine [PVP] (MW=200,000) with ethyl bromide. The QPVP contained 20% pyridinium units, as determined by infrared spectroscopy (i.e., 20% of the pyridine units attained a permanent positive charge through chemical reaction). Layers of the QPVP were deposited, in alternating sequence, with layers of PMAA (MW=150,000) on the surface of a Si crystal, following a modification of the procedure described in Example 1.

The QPVP and PMAA layers were deposited at pH 7 (0.01M phosphate buffer in D₂O) from 0.1 mg/ml solutions, with QPVP as the first layer. A 14-layer QPVP/PMAA film, having a thickness of 66 nm, with PMAA as the outermost layer, was produced.

The environmental pH of the film was then changed by contacting the surface of the QPVP/PMAA film with 0.01 M phosphate buffer solution at pH 5.5. At this pH, the net positive charge of the QPVP approximately doubled over the net positive charge at pH 7, indicating that 20% of the pyridine groups (based on PVP reacted) had become protonated. A solution of 0.5 mg/ml bromophenol blue dye in the same buffer was then brought into contact with the surface of the QPVP/PMAA film. The film was allowed to absorb the dye from solution for 1 hour. The bromophenol blue solution was then replaced by a pure buffer at pH 5.5 and dye content within the film was monitored as a function of time. There was no significant desorption of the dye from the QPVP/PMAA film for 1 hour (FIG. 11). The amount of dye absorbed was in 1:1 stiochiometric ratio with the amount of ionized groups in the QPVP/PMAA film.

The buffer solution was then replaced with a 0.1 mg/ml QPVP solution at pH 5.5. The release of the dye over the duration of one hour was measured and the results plotted (FIG. 5). The release rate was found to be limited by the rate of QPVP absorption. The amount of the dye released was proportional to the amount of QPVP absorbed. The results of the foregoing procedure are summarized below.

TABLE 4
Absorption and Release of Rhodamine
6G in Layered QVPV/PMAA Films

| | | Amount of | |
|---|---|---|---|
| | Percent of pyridine | bromophenol blue | Percent of the |
| pH | groups protonated | absorbed, mg/m² | dye released |
| 5.5 | 20 | 158 | 45 |

In accordance with the above procedures, the experiment was repeated using QVPV/PMAA films of other thicknesses. The results of these tests are summarized in Table 5.

TABLE 5
Absorption and Release of Rhodamine 6G in
Layered QVPV/PMAA Films of Various Thicknesses

| | | Percent of | Amount of | |
|---|---|---|---|---|
| | Film | pyridine | bromophenol | Percent of |
| | thickness, | groups | blue absorbed, | bromophenol |
| pH | nm | protonated | mg/m² | blue released |
| 5.5 | 49 | 20 | 32 | 38 |
| 5.5 | 58 | 20 | 52 | 42 |
| 5.5 | 66 | 20 | 158 | 45 |

### EXAMPLE 3

### Release and Absorption of Polymer in Response to Changes in Ionic Strength

The polymers QPVP and PMAA are the same as those described in Example 2. Alternating QPVP and PMAA layers were deposited at pH 9 from 0.1 mg/ml solutions in 0.01M borate buffer in D₂O. The deposition cycle started with QPVP and followed the protocol described in Example 1 hereinabove. A 10-layer QPVP/PMAA film, having thickness of 50 nm, with PMAA in the outermost layer, was produced. The film contained about 20 mg/m² of self-assembled PMAA and about 30 mg/m² of QPVP.

The layered film was then contacted with a buffer solution of pH 9 containing 0.4 M NaCl. Fast and complete release of the PMAA component occurred, with 95% of QPVP remaining at the surface, as illustrated in FIG. 6. The buffer solution was then replaced with 0.1 mg/ml of PMAA solution in a buffer at pH 9 and 0.3 M NaCl. This resulted in the binding of PMAA with the film, in the amount of 50% of the amount initially absorbed at low ionic strength conditions. After the ionic strength was further decreased to 0.1 M NaCl, an additional amount of PMAA became bound to the film, reaching a total amount of 96% of the initial amount of the self-assembled PMAA. The process could be repeated many times resulting in a controllable and reversible release and adsorption of PMAA as the ionic strength of the environmental solution was cycled between 0.4 M and 0.1 M NaCl.

In accordance with the above procedures, the experiments on the asymmetric release of PMAA were performed with the films composed of QPVP having other degrees of alkylation:

TABLE 6
Release and Absorption of PMAA from Layered QVPV/
PMAA Films of Various Thicknesses in Response to Changes
in Ionic Strength

| | | | | Amount of | |
|---|---|---|---|---|---|
| | | | | QPVP | Amount of |
| | QPVP | Film | Number of | released at | PMAA |
| | alkylation | thickness, | polymer | 0.4 M NaCl, | released at |
| pH | degree | nm | layers | mg/m² (%) | 0.4 M NaCl |
| 9 | 18 | 70 | 10 | 3 (5%) | 19 (97%) |
| 9 | 23 | 50 | 10 | 3 (5%) | 15 (98%) |
| 9 | 20 | 80 | 10 | 5 (8%) | 20 (98%) |

### EXAMPLE 4

### Adsorption and Release of IgG from Layered QPVP/PMAA Films

An 8-layer QPVP/PMAA film, having a thickness of 34 nm, with PMAA in the outermost layer, was produced following the procedure described in Example 3. The layer contained 12 mg/m² of self-assembled PMAA and about 22 mg/m² of QPVP.

The buffer solution in which the layered film was assembled was replaced by a buffer solution at pH 9 containing 0.4 M NaCl. Fast and complete release of PMAA component occurred, with 95% of QPVP remaining at the surface, similar to the release illustrated in FIG. 6. The saline buffer solution was then replaced with 0.1 mg/ml of Immunoglobuline (IgG) solution in a pH 9 buffer containing 0.2 M NaCl. This resulted in the binding of IgG in the film to an amount of 2.7 mg/m². After the film was contacted with a pH 9 buffer containing 0.01 M NaCl, an additional amount of IgG became bound to the film, to a final amount of 16 mg/m². The process was repeated a number of times, demonstrating a controllable and reversible release and adsorption of IgG as the environmental ionic strength was cycled between 0.4 M and 0.01 M NaCl. The results of this Example are illustrated in FIG. 7.

### EXAMPLE 5

### Release and Adsorption of RNAse from Layered PMAA/RNAse Films

A primer QPVP layer was deposited on a Si crystal substrate as described in Example 1. Alternating layers of PMAA and ribonuclease (RNAse) were deposited sequentially from 0.5 mg/ml solutions at pH 5.5. The deposition cycle started with a layer of PMAA, and subsequent layers were deposited following the protocol described in Example 1. A 10-layer of PMAA/RNAse film was produced, having a thickness of 15 nm, with RNAse in the outermost layer.

The buffer solution in which the film was produced was replaced by a buffer solution at pH 5.5 containing 0.3 M NaCl. Fast release of 5.8 mg/m² (70%) of the self-assembled RNAse was realized, with 80% of PMAA remaining at the surface, as shown in FIG. 8. The saline buffer solution was then replaced with 0.5 mg/ml RNAse solution in pH 5.5 buffer containing 0.1 M NaCl. This resulted in the binding of RNAse from solution to the substrate; to an amount of 5 mg/m². The layered film was then contacted with an 0.5 mg/ml solution of RNAse in pH 5.5 buffer (0.01 M buffer), with the result that additional RNAse became bound to the surface, to a final amount of 6.15 mg/m². The process was repeated a number of times, demonstrating a controllable and largely reversible (with a slight hysteresis) release and adsorption of RNAse as the ionic strength in the film's environment was cycled between 0.4 M NaCl and 0.01 M buffer at pH 5.5.

### EXAMPLE 6

### Release and Readsorption of PMAA from Layered QVPV/PMAA Films

The polymers QPVP and PMAA used in this example are described in Example 2. Alternating layers of QPVP and PMAA were deposited at pH 5 from 0.1 mg/ml solutions in 0.01M phosphate buffer in D₂O. The deposition cycle started with QPVP and followed the protocol described in Example 1 hereinabove. A 10-layer QPVP/PMAA film, having a thickness of 27 nm, with PMAA in the outermost layer, was produced. The layer contained 15 mg/m² of self-assembled PMAA and about 12 mg/m² of QPVP.

The buffer solution was then replaced by a 0.01 M phosphate buffer at pH 7. Fast release of 40% of the self-assembled PMAA was observed, while 98% of QPVP remained at the in the film. When the environmental pH of the film was further increased to pH 8 (0.01 M borate buffer), an additional 25% of the initial amount of PMAA was released from the film. The process was largely reversible (exhibiting a slight hysteresis), and 75% of the released PMAA was absorbed by the film when the pH of the film's environment was restored to pH 5. The process was repeated several times, demonstrating a controllable and reversible release and adsorption of PMAA as the environmental pH was cycled between pH 5, 7 and 8.

In accordance with the above procedures, the experiments on the asymmetric release and adsorption of PMAA were performed with another QPVP/PMAA film having 10 polymer layers and an initial thickness of 26 nm:

TABLE 7
Release and Readsorption of PMAA
from Layered QVPV/PMAA Films

| | |
|---|---|
| Release experiment | Readsorption |

| | Total | | of PMAA |
|---|---|---|---|
| | amount of | Total amount | Total amount |
| | PMAA in | of QPVP in | of PMAA in |
| | the film, | the film, | the film, |
| pH | mg/m² (%) | mg/m² (%) | mg/m² (%) |
| 5 | 15 (100%) | 12 (100%) | 11 (75%) |
| 7 | 9.2 (60%) | 11.8 (97%) | 8.3 (80%) |
| 8 | 5 (8%) | 11 (91%) | 0 |

### EXAMPLE 7

### Adsorption and Release of Lys from Layered PMAA/Lys Films

A primer QPVP layer was deposited on a Si crystal substrate as described in Example 1. Alternating layers of PMAA and lysozyme (Lys) were then deposited from 0.5 mg/ml solutions at pH 5, following the protocol described in Example 1 hereinabove. A 10-layer PMAA/Lys film was produced, having a thickness of 31 nm with Lys in the outermost layer.

The buffer solution was then replaced by a 0.5 mg/ml Lys solution in 0.01 M phosphate buffer at pH 7.5. Slow adsorption of an additional 17.5 mg/m² Lys was observed, to a total amount of 36.7 mg/m². When pH was then decreased to pH 5, release of Lys was observed and the amount of Lys absorbed decreased to 20 mg/m². When contacted with a 0.5 mg/ml Lys solution at pH=7.5, the film reabsorbed Lys to the initial concentration. The process was repeated a number of times, demonstrating a controllable and reversible release and readsorption of Lys as the environmental pH was cycled between pH 5 and pH 7.5.

In accordance with the above procedures, the experiments on the asymmetric release of Lys from the films were performed at different pH levels:

TABLE 8
Adsorption and Release of Lys from a Layered PMAA/Lys Film

| PH of | PH for | | | |
|---|---|---|---|---|
| multilayer | loading of | Amount of | Amount of Lys | |
| deposition | additional | Lys | additionally | Amount of lys |
| and Lys | amounts of | deposited at | absorbed at | released at pH |
| release | Lys | pH 5, mg/m2 | pH 6, mg/m2 | 5, mg/m2 |
| 5 | 6 | 20 | 8.5 | 8 |

The present invention presents several methods the controlled and/or reversible release of molecules which are absorbed in a self-assembled layered polymer film. The method for controlling the releasing low-molecular weight molecules by adsorption of oligomers or polymers, as discussed hereinabove and exemplified in Examples 1 and 2, goes beyond the known methods of controlling the release of such low-weight molecules, in that the method does not require the application of electric fields or changes in the pH of the film's environment. The release of the low-weight molecules from the film is proportional to the amount of charge provided to the film surface upon adsorption of the higher-weight oligomers or polymers. This release is sensitive merely to the presence of Molecule B on the outermost absorbed layer of the film.

In another embodiment, the present invention provides a method of selectively releasing oligomeric and polymeric molecules, such as natural and synthetic polypeptides, oligo- and polynucleotides or other molecules having plurality of charges, from a layered polymer film system, in response to variation in ionic strength. Such layered polymer films are stabilized by formation of a surface and can not be produced by simple adsorption of the macromolecular component from solution. Such surface films, which may be referred to as “surface sponges”, represent a new type of high-capacity material, which might be used in separations and release applications. As demonstrated in Examples 3-5, surface sponges are capable of absorbing and releasing large amounts of various macromolecular compounds from solution. These Examples also demonstrate that the absorption of macromolecular components within the sponges is reversible and can be modulated by changes in ionic strength. A wide variety of components, including synthetic and natural polyelectrolytes, such as proteins, heparin or oligonucleotides can be included and released from the films in a controlled way using this technique.

In yet another embodiment, the present invention provides methods of selectively releasing oligomeric and polymeric molecules, such as natural and synthetic polypeptides, oligo- and polynucleotides or other molecules having plurality of charges, from a layered polymer film system, in response to variations in the pH of the external solution. As demonstrated in Examples 6 and 7, the amounts of absorbed or released macromolecular components are large and are controlled by the total number of charges created into the multilayer when pH of the multilayer is varied. The absorption of macromolecular components is reversible. It is further demonstrated in Examples 1 and 2 that the loading and release capacity of the films can be easily manipulated by varying the film thickness, but is controlled by the changes in the net or excess charges in the film that result from changes in pH.

It is also noteworthy that, in addition to the types of polymers used in self-assembly of the alternating layers, other macromolecules, such as IgG or Lys, may be included as layers within the film. This will allow convenient one-step processes to produce high-capacity three-component layered films. Thus, the methods of the present invention may be extended to include and release a variety of components, including synthetic and natural polyelectrolytes, such as proteins, heparin or oligonucleotides, from the layered films.

Although the invention disclosed herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the invention as defined by the appended claims.

We claim:

1. Method for the controlled release of molecules from a film, comprising the steps of:
forming a multi-layer film comprising a polymer that can be modulated between an electrostatically charged state and an electrostatically uncharged state in response to a change in the pH of the film;
selecting a first molecule that has an electrostatic attraction to the polymer in a pH range within which the polymer has an excess charge;
adding a quantity of the first molecule to the multi-layer film;
adjusting the pH of the multi-layer film to a pH within a range of pH at which the polymer has an excess charge;
selecting a second molecule that adsorbs onto the multi-layer film within the range of pH at which the polymer has an excess charge; and
contacting the multi-layer film with a quantity of the second molecule at a pH within the range of pH at which the polymer has an excess charge, thereby causing a portion of said quantity of the first molecule to be released from the multi-layer film.

2. The method of claim 1, wherein the step of adding the quantity of the first molecule to the multi-layer film is performed concurrently with the step of forming the multi-layer film.

3. The method of claim 1, wherein the step of adding the quantity of the first molecule to the multi-layer film is performed after the step of forming the multi-layer film.

4. The method of claim 1, wherein the first molecule is a low molecular weight molecule selected from the group consisting of a dye and a bioactive agent.

5. The method of claim 1, wherein the second molecule has an electrostatic charge of the same sign as the polymer within the range of pH at which the polymer has the excess charge.

6. The method of claim 1, wherein the second molecule is a macromolecule selected from the group consisting of a polymer and an oligomer.

7. Method for the controlled release of molecules from a film, comprising the steps of:
forming a multi-layer film comprising a polymer, the layers of the multi-layer film adhering one to another through electrostatic interaction, said forming step being performed in a solution having a first ionic strength;
selecting a molecule that reversibly bonds with the multi-layer film;
adding a first quantity of the molecule to the multi-layer film; and
contacting the multi-layer film with a solution having a second ionic strength that is greater than the first ionic strength, thereby causing a second quantity of the molecule to be released from the multi-layer film.

8. The method of claim 7, further including the steps of contacting the multi-layer film with a solution of the molecule, wherein the solution has a third ionic strength that is lower than the second ionic strength, whereby a third quantity of the molecule reversibly bonds with the multi-layer film; and contacting the multi-layer film with a solution having a fourth ionic strength that is greater than the third ionic strength, thereby causing a fourth quantity of the molecule to be released from the multi-layer film.

9. The method of claim 7, wherein the step of adding the quantity of the molecule to the multi-layer film is performed concurrently with the step of forming the multi-layer film.

10. The method of claim 7, wherein the step of adding the quantity of the molecule to the multi-layer film is performed after the step of forming the multi-layer film.

11. The method of claim 7, wherein the molecule is selected from the group consisting of an oligomer and a polymer.

12. The method of claim 7, wherein the molecule is a bioactive agent.

13. The method of claim 7, wherein the polymer has moieties that can be modulated between an electrostatically charged state and an electrostatically uncharged state in response to a change in the pH of the film.

14. The method of claim 13, wherein the pH of the solution having the first ionic strength and the pH of the solution having the second ionic strength are substantially equal to each other.

15. Method for controlled release of macromolecules from a multi-layer film, comprising the steps of:
(a) selecting a polymer that can be modulated between an electrostatically charged state and an electrostatically uncharged state;
(b) selecting a macromolecule that bonds electrostatically to the polymer in its electrostatically charged state;
(c) forming a multi-layer film having sequential layers of the polymer and the macromolecule at a first pH at which the multi-layer film has a charge balance having a value of approximately one; and
(d) adjusting the pH of the multi-layer film so as to create a first excess charge of the multi-layer film, thereby releasing a first quantity of the macromolecule from the multi-layer film so as to restore the value of the charge balance to a value of approximately one.

16. The method of claim 15, further comprising the steps of: (e) adjusting the pH of the multi-layer film so as to create a second excess charge of the multi-layer film having a sign opposite to the sign of the first excess charge of the multi-layer film; and (f) contacting the multi-layer film with a solution of the macromolecule, whereby the multi-layer film takes up a second quantity of the macromolecule.

17. The method of claim 16, wherein steps (e) and (f) are performed before step (d).

18. The method of claim 16, wherein steps (d), (e) and (f) are performed in a sequence, further comprising the step of repeating steps (d), (e) and (f) in said sequence.

19. The method of claim 15, wherein the macromolecule is selected from the group consisting of a polymer and an oligomer.

20. The method of claim 15, wherein the macromolecule is a bioactive agent.
